(19) United States
(12) Patent Application Publication
(10) Pub. No.: US 2010/0210001 A1
(43) Pub. Date: Aug. 19, 2010

(54) BIOMASS CULTIVATING INSTALLATION AND METHOD

(76) Inventors: Ralf Seyfried, Meine (DE); Robert Frase, Hannover (DE); Jörg Nikolaus, Hannover (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/596,357

(22) PCT Filed: Apr. 2, 2008
(86) PCT No.: PCT/EP08/02618

§ 371 Date: Apr. 26, 2010

(30) Foreign Application Priority Data
Apr. 18, 2007 (DE) 102007018675.6

(51) Int. Cl. C12N 1/12; C12M 1/00; C12M 1/36

(52) U.S. Cl. 435/257.1; 435/292.1; 435/286.1

(57) ABSTRACT

The invention relates to a biomass cultivating installation (1) comprising a container (7) for receiving a solution containing biomass, at least one optical wave guide (8) guided in the container (7), for supplying light energy to the aqueous solution containing biomass, and a controllable light distributor (6) width is coupled to the optical wave guide (8) for the selective supply of light into selected regions of the container. The container (7) is split into segments comprising light radiation surfaces (9) that can be selectively coupled to the optical wave guide (5) by means of the light distributor (6). The optical wave guide (5) is coupled to a unit (3) for capturing sunlight and guiding the captured solar energy into the optical wave guide (5). A control unit (10) is provided for controlling the light distributor (6), in order to distribute the luminous powers available in the optical wave guide (5) to the light radiation surfaces (9) in such a way that another supply is carried out to a light radiation surface (9) when the at least one light radiation surface (9) supplied with luminous power from the optical wave guide (5) is supplied with a luminous intensity required for the significant mass growth of the biomass, and another luminous intensity is provided for supplying the other light radiation surface (9) also with a luminous intensity required for the significant mass growth of the biomass, and other light radiation surfaces (9) are disconnected such that a pre-determined minimum period of cumulated dark phases is provided according to the cumulated illumination interval of a segment.

The invention relates to a biomass cultivation plant having a container for holding a biomass-containing aqueous solution, having at least one optical waveguide introduced into the container for supplying light energy to the biomass-containing aqueous solution, and having a controllable light distributor, which is coupled to the light distributor for the selective supply of light to selected regions of the container.

The invention furthermore relates to a method for cultivating biomass, in particular algae, having a container, which is divided into a plurality of segments, for holding biomass-containing aqueous solutions and, for each segment, having in each case at least one light emission surface, which is coupled to an optical waveguide, in the container.

Solar energy plays a major role in the utilization of alternative raw materials in order to generate energy. Conventionally, solar energy is captured using collectors and used to heat a medium or converted to electric energy. The problem here is one of storing the generated energy since solar energy is often not available when the energy is needed.

U.S. Pat. No. 6,477,841 B1 describes a method for converting solar energy, which is stored by means of photosynthesis of algae, to electric energy.

The supply of light to the algae in a container poses a problem.

DE 39 33 486 A1 describes an appliance for cultivating aquatic organisms in sea water. It is proposed in this context to introduce vertically into the water a column having an apparatus attached thereto which collects solar rays and to guide the solar rays in the direction of the algae by means of optical waveguides.

KR 860000529 B proposes, for the purpose of supplying light into a photosynthesis reaction vessel using optical waveguides, to illuminate the optical waveguides by way of rotation of a light distributor sequentially one after the other.

NL 1027743 C discloses a method for stimulating algae growth in a reservoir by pumping water from a water source to a filter, where the biomass-containing water, as it is being passed through, is illuminated in a tube system in order to stimulate photosynthesis.

JP 2000060533 A describes an apparatus in which algae are stored in a container and light is guided to the container bottom via a light-guide plate.

JP 5292349 A proposes, for the purpose of promoting algae growth, to transfer sunlight energy from space to the earth by means of an electric wave in the gigahertz frequency range and to capture the electric radiation using a concave mirror and convert it to light energy.

WO 7900282 A1 describes a method for distributing a light beam in a photosynthesis medium. A strong light beam is guided via an optical waveguide and divided into a plurality of optical waveguide cables on which a number of emission surfaces are provided. It is thus possible to distribute light uniformly in the entire container.

In order to be able to realize the biomass-based energy generation even on a smaller scale for households, the useful luminous power must be captured efficiently and converted using photosynthesis for optimum growth of the biomass.

It is therefore an object of the present invention to provide an improved biomass cultivating installation and an improved method for cultivating biomass.

The object is achieved by way of the biomass cultivating installation of the type mentioned in the introduction in that the container is divided into segments, which in each case have light emission surfaces which can be coupled selectively to the optical waveguide via the light distributor, the optical waveguide is coupled to a unit for capturing sunlight and guiding the captured solar energy into the optical waveguide, and a control unit for actuating the light distributor is provided, which control unit is configured for distributing the luminous powers present in the optical waveguide to the light emission surfaces such that additional supply to a further light emission surface occurs if the at least one light emission surface, to which luminous power from the optical waveguide is supplied, is supplied with an intensity of illumination which is necessary for appreciable mass growth of the biomass and more luminous power is available for likewise supplying the further light emission surface with an intensity of illumination which is necessary for appreciable mass growth of the biomass, and that further light emission surfaces are switched off in a manner such that a predetermined minimum period of cumulative dark phases is provided as a function of the cumulative illumination period of a segment.

The available luminous power is used to best possible effect by the biocultivation installation according to the invention. It was recognized here that in order to achieve appreciable growth of biomass by photosynthesis, a minimum amount of light energy is needed. An appreciable increase in biomass only takes place once this minimum light energy is achieved.

It was furthermore found that the biomass growth rate decreases again after a certain amount of time. After uptake of a total luminous power, which exceeds a defined limit value as a function of the type of biomass, growth is saturated, as it were. It should therefore be ensured that in addition to the illumination phases necessary for photosynthesis, dark phases are also provided in which cell division takes place. The time period of the cumulative dark phases should be matched to the time period of the cumulative light phases.

For more efficiency it is advantageous to provide the biomass-containing aqueous solution not in a large volume but in container segments which are preferably separated from each other. The light energy supply to each of these container segments is then optimized by dividing the available light energy such that not too much and not too little light energy is introduced into the segments. For efficiency reasons it makes sense to step illumination of a segment completely for a period of time, if appropriate, rather than wasting light energy which does not exceed a minimum amount of light energy. Even if the biomass in a segment is already saturated with light energy, the light energy should be concentrated on other segments in which biomass growth with optimum efficiency by photosynthesis can be achieved.

It is particularly advantageous if the control unit is configured for cyclic light supply to a respective light emission surface with a sequence of light and dark phases. This is because it has been shown that biomass does not necessarily need a constant supply of light for photosynthesis. Rather, it is merely important to provide the minimum amount of light necessary during the illumination and sufficient luminous power over the time. Owing to the light and dark phases, the available light energy during a light phase can always be concentrated on selected segments and a minimum amount of light energy can be provided for the segments. By alternating light and dark phases it is also possible to supply light energy to the segments of the container in a relatively uniform fashion.

The control unit is configured for regulating the illumination intensity of individual light emission surfaces as a function of the available luminous power and of the illumination intensity necessary for appreciable mass growth of the biomass by matching the pulse width of the cyclic light supply to the respective light emission surfaces.

Removal of the produced biomass for further processing and utilization as energy carriers is preferably done using harvesting devices which are arranged in the segments of the containers. Said harvesting devices are coupled to the light emission surfaces in order to remove biomass which adheres to the light emission surface, and so not just to harvest the biomass but also simultaneously clean the light emission surfaces. Such a harvesting device can for example have wiping elements which are movable on the surface of the light emission surface and are designed for example in the manner of a screen wiper.

The wiping elements can be mounted, for example, on a movable carrier and have rubber lip profiles which face in the direction of the light emission surface. The carrier can here be movable perpendicularly from the top downward in a respective segment.

Suction openings are preferably provided in the container at the bottom of the segments for removing biomass, which collects on the bottom, by suction. The suction openings can then be communicatively connected to a tube system. At least one separator for removing biomass is then connected to the tube system. A dryer for drying the biomass and a pressing apparatus for compressing the dried biomass, for example into pellets or bricks, can then be connected to said separator which is preferably controlled. These pellets or bricks can then be passed into a pellet stove.

In one embodiment, in each case one light-guide fabric web can be introduced into each of the container segments, which fabric web is coupled to the light distributor for injecting light. The light-guide fabric web is mounted such that it is movable for harvesting the biomass, for example on transport rollers, such that one region of the light fabric web to which light energy is applied is supplied to the harvesting apparatus, while light is again applied to another region which is used for the further biomass cultivation. Such a light-guide fabric web can be realized by way of example as an endless web.

The light distributor can, for example, have a distribution unit which has at least one movably arranged mirror surface or lens which can be manipulated using a drive unit. The mirror surfaces or lenses are then coupled to ac least one supply waveguide for supplying light energy from the sun capturing unit and to a plurality of removal waveguides, which are guided to the respective segments, in order to selectively transfer light energy—depending on the position of the mirror surfaces or lenses—from supply waveguides to selected removal waveguides.

In another advantageous embodiment, the light distributor has an actuator which is connected to an optical waveguide for supplying light energy from the sunlight capturing unit and is configured for the rotation or movement of the exit end face of the supply waveguide to an at least one entry end face of at least one selected removal waveguide which is guided to a respective segment. The removal waveguides are arranged here such that their entry end faces lie opposite the exit end face of the supply waveguide, which is moved parallel to a plane defined by the entry end faces of the removal waveguides.

At least one concave mirror can be used as the sunlight capturing device. However, it is particularly advantageous if the device for capturing sunlight has at least one light collector which has a coupling-in region, toward which the optical waveguide which is provided for passing on the light energy to the light distributor is orientated.

The biomass cultivating installation can have a heat exchanger and/or a heat pump in order to convert excess hot or cold energy of the biomass installation, in particular of the container, the light collector and/or light distributor, and to supply it for further utilization. For example the heat freed during cooling of the biomass installation can be used to heat service water, for example.

It is particularly advantageous if the biomass cultivating installation is coupled to a combustion device for the biomass produced and if exhaust gases and/or combustion residues of the combustion device are returned to the container of the biomass cultivating installation. It is possible in this manner to fertilize the nutrient solution for the biomass.

It is advantageous if gaseous, liquid and/or solid combustion residues are stored temporarily. It is thus possible to match varying quantities of requirement and production to each other without over-fertilizing the nutrient medium. The biomass cultivating installation can then also form a closed system where all the exhaust gases and combustion residues are returned.

The object is furthermore achieved by the method of the type mentioned in the introduction by way of the following steps:

a) capturing solar light,

b) guiding the captured solar light into an optical waveguide,

c) measuring the luminous power available in the optical waveguide and

d) distributing the available luminous power from the optical waveguide to selected light emission surfaces such that additional supply to a further light emission surface occurs if the at least one light emission surface, to which luminous power from the optical waveguide is supplied, is supplied with an intensity of illumination which is necessary for appreciable mass growth of the biomass and more luminous power is available for likewise supplying the further light emission surface with an intensity of illumination which is necessary for appreciable mass growth of the biomass, and that further light emission surfaces are switched off in a manner such that a predetermined minimum period of cumulative dark phases is provided as a function of the cumulative illumination period of a segment.

For the further utilization of the produced biomass as fuel it is advantageous to subject them to frothing and to thus include air or gas in the biomass, which is processed further into fuel pellets or fuel bricks, for adjusting the calorific value.

It is furthermore advantageous to add additives to the produced biomass before, during or after the drying of the separated biomass, the pulverization of the dried biomass, the frothing of the biomass and/or the pressing of the dried biomass into fuel pellets or fuel bricks. This can likewise be used, by way of example, for regulating the calorific value.

The subclaims describe advantageous embodiments.

The invention will be explained in more detail below with reference to the appended drawing, in which:

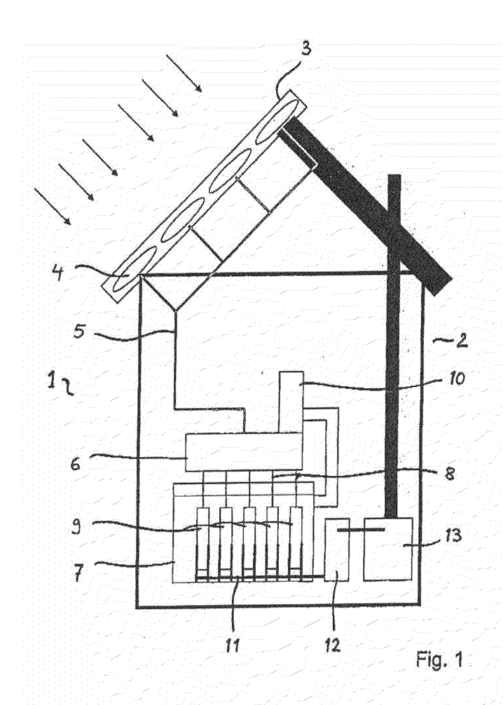

FIG. 1 shows a sketch of a biomass cultivating installation.

FIG. 1 shows a biomass cultivating installation 1, which is installed in a house for supplying the household. Sunlight is captured using a unit 3 for capturing to sunlight. This unit 3 can be a light collector, for example, in which converging lenses, such as Fresnel lenses 4, are arranged on a surface, with the coupling-in regions of optical waveguides 5 being arranged in the foci of said converging lenses in order to inject the captured solar energy into the optical waveguides 5.

These optical waveguides 5, formed, for example, from fiber-optic cable, are guided into a light distributor 6 in order to guide, in a controlled fashion, the luminous power from there into individual segments of a container 7 and to illuminate biomass-containing aqueous solution, which is contained in the container 7, in order to stimulate a photosynthesis process. To this end, removal waveguides 8 are used, which emerge from the light distributor 5 and are connected to respectively associated light emission surfaces 9 which are arranged in the individual segments.

For actuating the light distributor 8, a control unit 10 is provided which controls the light supplied to the light emission surfaces 9 on the basis of the available luminous power, which is measured using appropriate sensors. The light emission surfaces 9 are supplied by applying light to a light emission surface 9 only if a minimum amount of light energy necessary for cell division of the biomass can be provided. The luminous power available in the optical waveguide 5 is thus bundled such, and distributed to the light emission surfaces 9, that each of the light emission surfaces 9, to which luminous power is applied, emits a minimum amount of light energy necessary for the cell division of the biomass. This minimum amount of light energy is dependent on the type of biomass which propagates with the aid of photosynthesis, such as bacteria, plankton, lichens, bryophytes, aquatic plants, algae, in particular blue-green algae, etc. By way of example, fresh water or sea water, if appropriate with the addition of nutrients, can be used as the aqueous solution.

The control unit 10 can be used in conjunction with the light distributor 6 for controlling light-dark phases at the individual light emission surfaces 9, which can serve for stimulating cell division.

Moreover, the control unit 10 can control the averaged illumination intensity of the light emission surfaces 9 by way of distribution of the light energy to the optical waveguides 5, which are coupled to the light emission surfaces 9.

This distribution of the light energy in the light distributor 6 can be realized, for example, by pulse-width modulation (PWM) in conjunction with a digital controller, wherein an optical waveguide 5—which leads to the light emission surfaces 9—is, if possible, always illuminated in order to achieve as high a degree of efficiency as possible.

If the light pulse frequency at the light emission surfaces 9 is sufficiently great here, some types of algae behave, for example, as in the case of continuous illumination at the same averaged illumination intensity, it is likewise conceivable to control the illumination intensity in an analog fashion by way of beam splitters in the light distributor 6.

It is important that the control unit 10 in conjunction with the light distributor 6 ensures that as much of the container 7, which forms a bioreactor, as possible is operated at its optimum operating point in order to achieve as high a degree of efficiency during the cell division as possible. The control unit 10 takes into account a hysteresis behavior during the growth of biomass, where an appreciable growth occurs only above a light energy which is the minimum amount necessary for cell division. Any major cell division takes place only once this minimum amount of light energy is reached. It should also be taken into account, however, that, light might be necessary for the growth of the biomass, but that the biomass, in particular algae, only undergo further cell division once it is dark. Thus a dark phase is also necessary, which must likewise be taken into account by the control unit 10.

The control unit 10 furthermore serves for controlling the introduction of nutrient into the biomass-containing aqueous solution and the harvesting operation.

A harvesting device 11 is provided on the bottom of the container 7 in order to remove the biomass from the container and to transfer it to a post-processing device 12. The post-processing device 12 can be, in particular, a dryer and a pellet press for pressing the dried biomass into pellets. The pellets produced from the biomass are then transferred to a pellet stove 13 for their combustion and for the supply of energy.

It is also possible to use, instead of the illustrated light collector 3 with Fresnel lenses 4, optionally, other converging lenses or concave mirror arrangements. The light collector 3 is then mounted on vertical and/or horizontal surfaces and can optionally have a means for adjusting the angle of incidence and in particular a tracking means in order to adjust the angle of the light collector always in an optimum fashion to the position of the sun. In order to avoid overheating or to shut off the installation, an automatic screening means can preferably be provided on a light collector 3, for example designed in the form of blinds. Furthermore, a cleaning apparatus can be provided in order to clean the light collector 3 in the case of soiling or snow etc.

The optical waveguide 5 serves for transporting the light energy, with only the luminous power needing to be passed on, but not the thermal energy generated by the irradiation. The optical waveguide 5 can therefore be designed as a fiber-optical cable or plastic cable. The use of tubes which are reflective on the inside or other mirror systems is also conceivable.

Furthermore advantageous is the integration of a breakage detector into the optical waveguide 5 in order to be able to detect damage to the optical waveguide 5.

Light panels, fiber-optical cables, round bars etc. can be used as luminous emission surfaces. Conceivable is also, by way of example, the use of a light-guide fabric web, which is suspended in a segment of the container 7 in the form of a mat—which is preferably of the endlessly circumferential type—and is coupled to the light distributor 6 such that luminous power is injected into the light-guide fabric web and coupled out at the surface of the light-guide fabric web. The light-guide fabric web can in that case be movably mounted in the container on rollers in order to guide individual regions of the light-guide fabric web to a harvesting device 11.

The harvesting device 11 can have, by way of example, a number of wiper blades, which are movably arranged on one of more holders and are movable on the light emission surfaces 9 in order to scrape off the biomass which collects on the light emission surfaces 9 and supply it to the harvesting device 11.

It is also conceivable, however, to harvest the biomass by repumping the biomass-containing aqueous solution and guiding the solution through a filter. To this end, suction openings should be provided in the individual segments on the bottom of the container, which openings are coupled to a corresponding pump-action extraction tube system.

The biomass cultivating installation 1 has the advantage chat it can be controlled easily using the control unit 10. Remote maintenance and monitoring, for example via telephone, internet or radio data applications, is also possible here.

It is possible with the biomass cultivating installation 1 to successfully convert sunlight into thermal energy and/or electric energy over the whole year via a cost-effective and loss-free temporary store for the collected energy in the form of a solid fuel substrate from the biomass obtained. The light and nutrient input and the temperature conditions can be matched in an optimum fashion via the control unit 10 and the light distributer 6 to the growth resources, i.e. the type of biomass, in order to optimize the yield and volume requirement. In particular, the energy accumulation can be decoupled in a temporary manner from the energy removal. Sunlight can be converted to solid fuel over the entire year, but said solid fuel can be used as required. The result is an advantage in efficiency.

The biomass cultivating installation 1 can be operated autonomously as shown in households. Conceivable is also, however, operation of networked systems in a coupled energy supply network. In this case, the method can be realized completely autonomously with the aid of remote monitoring by a central system.

Due to the physical decoupling of the light collector 3 from the remainder of the biomass cultivating installation 1 by the low-loss light transport in the optical waveguide 5 without the necessity of the thermal energy transport, extensive freedom of design when realizing the biomass cultivating installation 1 and optimum matching to various local conditions is possible.

The biomass used can be, for example, algae, such as Chlorella pyrenoidosa. In the case of illumination over 24 hours and a light energy of 10 kLux, 9 to 11 divisions at a temperature of 30 to 35° are possible. The light requirement of 10 kLux approximately corresponds to a tenth of the maximum daylight current.

1. A biomass cultivating installation (1) having a container (7) for holding a biomass-containing aqueous solution, having at least one optical waveguide (8) introduced into the container (7) for supplying light energy to the biomass containing aqueous solution, and having a controllable optical waveguide (5), which is coupled to the optical waveguide (8) for the selective supply of light to selected regions of the container (7), characterized in that the container (7) is divided into segments, which in each case have light emission surfaces (9) which can be coupled selectively to the optical waveguide (5) via the light distributor (6), the optical waveguide (5) is coupled to a unit (3) for capturing sunlight and guiding the captured solar energy into the optical waveguide (5), and a control unit (10) for actuating the light distributor (6) is provided, which control unit is configured for distributing the luminous powers present in the optical waveguide (5) to the light emission surfaces (9) such that additional supply to a further light emission surface (9) occurs if the at least one light emission surface (9), to which luminous power from the optical waveguide (5) is supplied, is supplied with an intensity of illumination which is necessary for appreciable mass growth of the biomass and more luminous power is available for likewise supplying the further light emission surface (9) with an intensity of illumination which is necessary for appreciable mass growth of the biomass, and that further light emission surfaces (9) are switched off in a manner such that a predetermined minimum period of cumulative dark phases is provided as a function of the cumulative illumination period of a segment.

2. The biomass cultivating installation (1) as claimed in claim 1, characterized in that the control unit (10) is configured for cyclic light supply to a respective light emission surface with a sequence of light and dark phases.

3. The biomass cultivating installation (1) as in claim 2, characterized in that the control unit (10) is configured for regulating the illumination intensity of individual light emission surfaces (9) as a function of the available luminous power and of the illumination intensity necessary for appreciable mass growth of the biomass by matching the pulse width of the cyclic light supply to the respective light emission surfaces (9)

4. The biomass cultivating installation (1) as claimed in claim 1, characterized by a harvesting device (11) which is arranged in the segments of the container (7) and coupled to the light emission surfaces (9) such that biomass, which adheres to the light emission surface (9) after the harvesting device (11) is actuated and the biomass is harvested, is removed.

5. The biomass cultivating installation (1) as claimed in claim 4, characterized in that the harvesting device (11) has wiping elements which are movable on the surface of the light emission surface (9)

6. The biomass cultivating installation (1) as claimed in claim 5, characterized in that the wiping elements have rubber lip profiles which are mounted on a movable carrier and face in the direction of the light emission surface (9).

7. The biomass cultivating installation (1) as claimed in claim 6, characterized in that the carrier is movable perpendicularly from the top downward in a respective segment.

8. The biomass cultivating installation (1) as claimed in claim 1, characterized in that suction openings are provided in the container at the bottom of the segments for removing biomass, which collects on the bottom, by suction.

9. The biomass cultivating installation (1) as claimed in claim 8, characterized in that the suction openings can be communicatively connected to a tube system to which a separator for removing biomass is connected.

10. The biomass cultivating installation (1) as claimed in claim 1, characterized in that in each case one light-guide fabric web is introduced into each container segment, which fabric web is coupled to the light distributor (6) for injecting light and is mounted such that it is movable for harvesting the biomass.

11. The biomass cultivating installation (1) as claimed in claim 1, characterized in that the light distributor (6) has a distribution unit which has at least one mirror surface which is arranged such that it can be rotated by a drive unit or has at least one lens, which is coupled to at least one supply waveguide (5) for supplying light energy from the sunlight capturing unit and to a plurality of removal waveguides (8), which are guided to the respective segments, in order to selectively transfer light energy from supply waveguides to selected removal waveguides (8)

12. The biomass cultivating installation (1) as claimed in claim 1, characterized in that the light distributor (6) has an actuator which is connected to an optical waveguide (5) for supplying light energy from the sunlight capturing unit and is configured for the rotation or movement of the exit end face of the supply waveguide (5) to an at least one entry end face of at least one selected removal waveguide (8) which is guided to a respective segment, wherein the removal waveguides (8) are arranged such that their entry end faces lie opposite the exit end face of the supply waveguide (5)

13. The biomass cultivating installation (1) as claimed in claim 1, characterized by a controlled separator which is configured for removing biomass from the container segments, a dryer, which is connected to the output of the separator, for drying the biomass and a pressing apparatus, which is connected to said dryer, for compressing the dried biomass.

14. The biomass cultivating installation (1) as claimed in claim 1, characterized by concave mirrors as device (3) for capturing sunlight.

15. The biomass cultivating installation (1) as claimed in claim 1, characterized in that the device (3) for capturing sunlight has at least one light collector which has a coupling-in region for passing on the light energy to the light distributor (6) by way of the optical waveguide (5) provided and the optical waveguide (5) is orientated towards the coupling in region.

16. The biomass cultivating installation (1) as claimed in claim 15, characterized in that the light collector has one or more converging lenses, in particular Fresnel lenses, and/or one or more concave mirrors.

17. The biomass cultivating installation (1) as claimed in claim 1, characterized in that the biomass cultivating installation is configured for the propagation of algae.

18. The biomass cultivating installation (1) as claimed in claim 1, characterized in that the biomass cultivating installation has a heat exchanger and/or a heal pump for the conversion and further utilization of excess hot or cold energy of the biomass installation, in particular of the container, the light collector and/or light distributor (6).

19. The biomass cultivating installation (1) as claimed in claim 1, characterized in that the biomass cultivating installation is coupled to a combustion device for the biomass produced and exhaust gases and/or combustion residues of the combustion device are returned to the container of the biomass cultivating installation.

20. The biomass cultivating installation (1) as claimed in claim 19, characterized in that the biomass cultivating installation forms a closed system and is configured for the return of all the exhaust gases and combustion residues.

21. The biomass cultivating installation (1) as claimed in claim 19, characterized by a temporary storage means for gaseous, liquid and/or solid combustion residues.

22. A method for cultivating biomass, in particular algae, in a container (7), which is divided into a plurality of segments, for holding biomass containing aqueous solutions and, for each segment, having in each case at least one light emission surface (9), which is coupled to an optical waveguide (8), in the container (7) having the following steps:
a) capturing solar light,
b) guiding the captured solar light into the optical waveguide (5),
characterized by
c) measuring the luminous power available in the optical waveguide (5) and
d) distributing the available luminous power from the optical waveguide (5) to selected light emission surfaces (9) such that additional supply to a further light emission surface (9) occurs if the at least one light emission surface (9), to which luminous power from the optical waveguide (5) is supplied, is supplied with an intensity of illumination which is necessary for appreciable mass growth of the biomass and more luminous power is available for likewise supplying the further light emission surface (9) with an intensity of illumination which is necessary for mass growth of the biomass, and that light emission surfaces (9) are switched off in a manner such that a predetermined minimum period of cumulative dark phases is provided as a function of the cumulative illumination period of a segment.

23. The method as claimed in claim 22, characterized by a cyclic light supply of the luminous power to selected light emission surfaces (9) with a sequence of light and dark phases.

24. The method as claimed in claim 23, characterized by regulating the illumination intensity of individual light emission surfaces (9) as a function of the available luminous power and of the illumination intensity necessary for appreciable mass growth of the biomass by matching the pulse width of the cyclic light supply to the respective light emission surfaces (9)

25. The method as claimed in claim 22, characterized by wiping off the light emission surface (9) using a wiping apparatus for cleaning and removing adhering biomass and removing the biomass on the wiping apparatus by suction and/or separating the biomass which has collected on the bottom.

26. The method as claimed in claim 22, characterized by separating the biomass from selected segments, drying the separated biomass and pulverizing the dried biomass or pressing the dried biomass into fuel pellets or fuel bricks.

27. The method as claimed in claim 22, characterized by disposing of the biomass, which has absorbed and converted carbon dioxide (CO2) from the atmosphere during the propagation, for reducing the carbon dioxide content in the atmosphere.

28. The method as claimed in claim 22, characterized by converting and further utilizing excess hot or cold energy of the biomass cultivating-installation (1), in particular of the container, the light collector and/or light distributor (6), by means of a heat exchanger and/or heat pump.

29. The method as claimed in claim 22, characterized by returning exhaust gases and/or combustion residues of a combustion device, which is coupled to the biomass cultivating installation (1), for the biomass produced into the container of the biomass cultivating installation (1)

30. The method as claimed in claim 29, characterized by returning all the exhaust gases and combustion residues so that the biomass cultivating installation forms a closed system.

31. The method as claimed in claim 29, characterized by temporarily storing gaseous, liquid and/or solid combustion residues.

32. The method as claimed in claim 22, characterized by subjecting the biomass to frothing for adjusting the calorific value of the fuel pellets or fuel bricks, in particular for the inclusion of air.

33. The method as claimed in claim 22, characterized by adding additives to the produced biomass, for example for regulating the calorific value, before, during or after the drying of the separated biomass, the pulverization of the dried biomass, the frothing of the biomass and/or the pressing of the dried biomass into fuel pellets or fuel bricks.

34. The method as claimed in claim 22, characterized by fertilizing the biomass as a function of a determined operating state of the biomass cultivating installation (1), for example as a function of the measured carbon dioxide content in the nutrient medium of the biomass.
